(19) United States
(12) Patent Application Publication
(10) Pub. No.: US 2015/0289853 A1
(43) Pub. Date: Oct. 15, 2015

(54) ULTRASONIC DIAGNOSIS APPARATUS

(71) Applicant: SAMSUNG ELECTRONICS CO., LTD., Suwon-si (KR)

(72) Inventors: Kyungil CHO, Seoul (KR); Jihoon BANG, Yongin-si (KR); Jong Keun SONG, Yongin-si (KR)

(73) Assignee: SAMSUNG ELECTRONICS CO., LTD., Suwon-si (KR)

(21) Appl. No.: 14/616,049

(22) Filed: Feb. 6, 2015

(30) Foreign Application Priority Data
Apr. 15, 2014 (KR) 10-2014-0044639

(51) Int. Cl. A61B 8/00; A61B 8/14

(52) U.S. Cl. CPC A61B 8/546; A61B 8/14; A61B 8/4427; A61B 8/462

(57) ABSTRACT

An ultrasonic diagnosis apparatus includes a transducer configured to transmit ultrasonic waves to an object and receive echo signals, a beamformer configured to generate output signals by performing beamforming on the echo signals, a port configured to engage with a portable terminal, and a controller configured to control an ultrasonic image to be displayed on the portable terminal by transmitting the output signals to the portable terminal engaged with the port.

### CROSS-REFERENCE TO RELATED APPLICATION

This application claims priority from Korean Patent Application No. 10-2014-0044639, filed on Apr. 15, 2014, in the Korean Intellectual Property Office, the disclosure of which is incorporated herein by reference in its entirety.

### BACKGROUND

1. Field

Apparatuses and methods consistent with exemplary embodiments relate to an ultrasonic diagnosis apparatus that diagnoses diseases.

2. Description of the Related Art

An ultrasonic diagnosis apparatus is an apparatus that transmits ultrasonic waves toward a target portion inside an object and receives reflected ultrasonic echo signals to acquire tomographic images or blood stream images of the target portion, e.g., soft tissues, in a noninvasive manner.

Compared to other medical image diagnosis apparatuses such as an X-ray diagnosis apparatus, an X-ray computed tomography (CT) scanner, a magnetic resonance imaging (MRI) apparatus, a nuclear medicine diagnosis apparatus, and the like, the ultrasonic diagnosis apparatus is compact and inexpensive, displays ultrasonic images in real-time, and has higher safety without exposure to X-rays. Therefore, the ultrasonic diagnosis apparatus has been widely used for cardiac, abdominal, urinary, and obstetrical diagnoses.

The ultrasonic diagnosis apparatus emits ultrasonic waves to the object and receives ultrasonic echo signals reflected from the object to generate an ultrasonic image.

### SUMMARY

One or more exemplary embodiments provide an ultrasonic diagnosis apparatus using resources of a portable terminal. In addition, one or more exemplary embodiments provide an ultrasonic diagnosis apparatus including a configuration in which heat generated from the ultrasonic diagnosis apparatus can be emitted.

Additional aspects of the invention will be set forth in part in the description which follows and, in part, will be obvious from the description, or may be learned by practice of the invention.

In accordance with an aspect of an exemplary embodiment, an ultrasonic diagnosis apparatus includes: a transducer that radiates ultrasonic waves to an object and receives echo signals; a beamformer that generates output signals by performing beamforming on the echo signals; a port that docks a portable terminal; and a controller that controls an ultrasonic image to be displayed in the portable terminal by transmitting the output signals to the docked portable terminal.

Here, the controller may control the portable terminal to receive the output signals, and control the portable terminal to perform image-processing on the received output signals.

Also, the controller may control the portable terminal so that the ultrasonic image is displayed on a display provided in the portable terminal.

Also, the output signals may be transmitted to the portable terminal through the port.

Also, the output signals may be transmitted to the portable terminal in a wireless communication scheme.

Also, the ultrasonic diagnosis apparatus may further include an upper housing in which the port is provided; and a lower housing that receives the transducer, the beamformer, and the controller. Here, a space in which the portable terminal is seated may be formed by combining the upper housing and the lower housing.

Also, the upper housing may be replaceable depending on a terminal port of the portable terminal.

Also, the ultrasonic diagnosis apparatus may further include a fixer that fixes the docked portable terminal.

Also, the ultrasonic diagnosis apparatus may further include a heat radiator that emits heat generated by driving of the ultrasonic diagnosis apparatus to the outside.

Also, the heat radiator may emit the heat generated by the driving to the outside in a heat conduction scheme.

Also, the heat radiator may include a cooling fan, and emit the heat generated by the driving to the outside by rotating the cooling fan.

Also, power for driving the ultrasonic diagnosis apparatus may be supplied from the portable terminal through the port.

Also, the ultrasonic diagnosis apparatus may further include a power supply unit that supplies power for driving the ultrasonic diagnosis apparatus.

Also, the portable terminal may be docked so as to be inclined at a predetermined angle with respect to the transducer.

Also, the transducer may be located on an opposite side of a surface on which the portable terminal is docked.

Also, the controller may control operations of the ultrasonic diagnosis apparatus in accordance with control signals received from the portable terminal.

### BRIEF DESCRIPTION OF THE DRAWINGS

The above and/or other aspects will become more apparent by describing certain exemplary embodiments with reference to the accompanying drawings, in which:

FIG. 1 is a perspective view showing a medical system in accordance with an exemplary embodiment;

FIG. 2 is a cross-sectional view showing a medical system in accordance with an exemplary embodiment;

FIG. 3 is a perspective view showing an ultrasonic diagnosis apparatus in accordance with an exemplary embodiment;

FIG. 4 is a perspective view showing a portable terminal in accordance with an exemplary embodiment;

FIG. 5 is a control block diagram showing a medical system in accordance with an exemplary embodiment;

FIG. 6 is a cross-sectional view showing a medical system in accordance with an exemplary embodiment;

FIG. 7 is a perspective view showing a front side of a medical system in accordance with an exemplary embodiment;

FIG. 8 is a perspective view showing a rear side of a medical system in accordance with an exemplary embodiment;

FIG. 9 is a cross-sectional view showing a medical system in accordance with an exemplary embodiment;

FIG. 10 is a control block diagram showing a medical system in accordance with an exemplary embodiment;

FIG. 11 is a perspective view showing an example of a heat radiator;

FIG. 12 is a schematic cross-sectional view showing a medical system in accordance with an exemplary embodiment.

### DETAILED DESCRIPTION

Certain exemplary embodiments are described in greater detail below with reference to the accompanying drawings, wherein like reference numerals refer to like elements throughout. Prior to the description, it should be understood that the terms used in the specification and the appended claims should not be construed as limited to general dictionary meanings, but interpreted based on the meanings and concepts corresponding to technical aspects of the disclosure on the basis of the principle that the inventor is allowed to define terms appropriately for the best explanation. Therefore, the description proposed herein is merely an example for the purpose of illustration only, not intended to limit the scope of the disclosure, and thus it should be understood that other equivalents and modifications could be made thereto without departing from the spirit and scope of the disclosure.

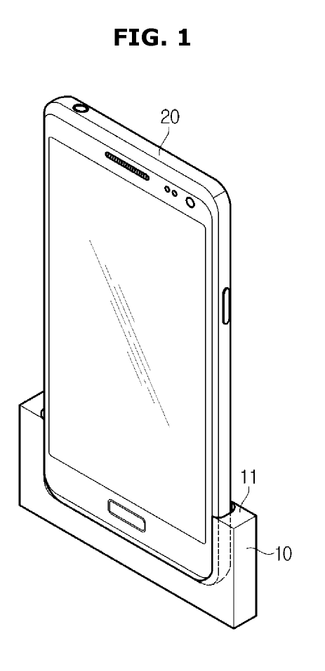

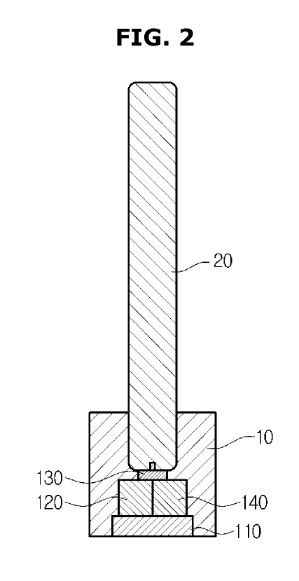

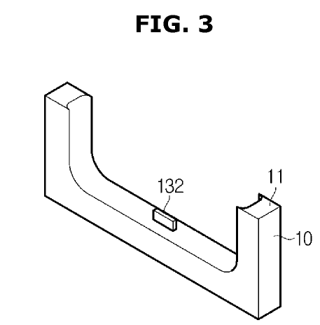

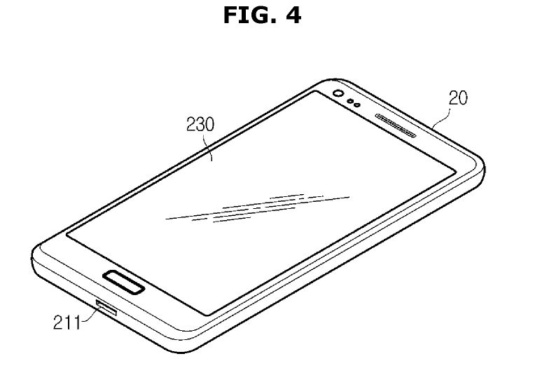

FIG. 1 is a perspective view showing a medical system in accordance with an exemplary embodiment, FIG. 2 is a cross-sectional view showing a medical system in accordance with an exemplary embodiment, FIG. 3 is a perspective view showing an ultrasonic diagnosis apparatus 10 in accordance with an exemplary embodiment, and FIG. 4 is a perspective view showing a portable terminal in accordance with an embodiment.

Referring to FIGS. 1 to 4, a medical system may include an ultrasonic diagnosis apparatus 10 and a portable terminal 20. In this instance, the medical system may provide ultrasonic images to users based on the ultrasonic diagnosis apparatus 10 and the portable terminal 20.

The ultrasonic diagnosis apparatus 10 may transmit ultrasonic waves to an object, and receive echo signals reflected from the object to thereby generate ultrasonic images. In this instance, the ultrasonic diagnosis apparatus 10 may generate the ultrasonic images using hardware and/or software included in the portable terminal 20.

For this, the ultrasonic diagnosis apparatus 10 and the portable terminal 20 may be docked together in various methods. For example, as shown in FIG. 3, the ultrasonic diagnosis apparatus 10 may have a port 132 in the form of a plug that protrudes to the outside, and as shown in FIG. 4, the portable terminal 20 may have a terminal port 211 in the form of a socket.

That is, the ultrasonic diagnosis apparatus 10 and the portable terminal 20 may be docked together by coupling the port 132 of the ultrasonic diagnosis apparatus 10 and the terminal port 211 of the portable terminal 20. In this manner, the ultrasonic diagnosis apparatus 10 may generate the ultrasonic images using resources of the docked portable terminal 20, thereby reducing a size of the ultrasonic diagnosis apparatus 10.

In addition, the ultrasonic diagnosis apparatus 10 may generate the ultrasonic images using hardware of the portable terminal 20, thereby reducing production costs of the ultrasonic diagnosis apparatus 10.

In addition, the portable terminal 20 and the ultrasonic diagnosis apparatus 10 are integrally used in a state in which they are docked together, whereby it is possible to more readily perform operations of the ultrasonic diagnosis apparatus 10. More specifically, a user of the ultrasonic diagnosis apparatus 10 may acquire the ultrasonic images by readily operating the ultrasonic diagnosis apparatus 10 and the portable terminal 20 with one hand. Thus, it is possible to more readily observe the ultrasonic images in times of emergency or the like, and take actions while observing the ultrasonic images.

In addition, the ultrasonic diagnosis apparatus 10 may further include a fixer 11 for fixing the docked portable terminal 20. For example, as shown in FIGS. 1 and 3, the fixer 11 forms a space in which the docked portable terminal 20 can be installed, and the docked portable terminal 20 is seated on the fixer 11 and fixed to the ultrasonic diagnosis apparatus 10.

Here, the fixer 11 is integrally formed with a housing of the ultrasonic diagnosis apparatus 10 to form a space in which the portable terminal 20 is fixed. In an exemplary embodiment, the fixer 11 may be coupled to a side of the housing of the ultrasonic diagnosis apparatus 10, according to an exemplary embodiment.

In addition, a surface on which the fixer 11 and the portable terminal 20 are brought into contact with each other is coated with a material having a high frictional force, and therefore the portable terminal 20 can be more firmly coupled to the ultrasonic diagnosis apparatus 10.

The portable terminal 20 is docked with the ultrasonic diagnosis apparatus 10 so that the ultrasonic diagnosis apparatus 10 can generate the ultrasonic images. For example, the portable terminal 20 may receive data from the ultrasonic diagnosis apparatus 10 and process the received data to transmit the processed data to the ultrasonic diagnosis apparatus 10 again, or generate the ultrasonic images based on the data received from the ultrasonic diagnosis apparatus 10 to output the generated ultrasonic images.

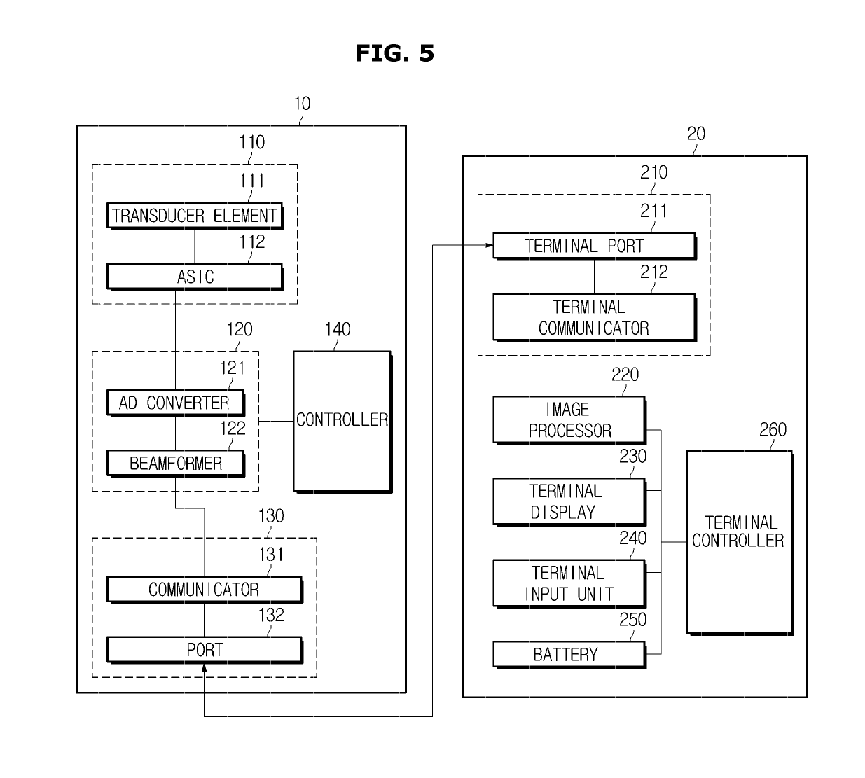

FIG. 5 is a control block diagram showing a medical system in accordance with an exemplary embodiment. Hereinafter, the medical system will be described in more detail with reference to FIGS. 1 to 5.

The ultrasonic diagnosis apparatus 10 may include a transducer 110, a beamformer 120, a communicator 130, and a controller 140.

The transducer 110 may include at least one transducer element 111 and an application specific integrated circuit (ASIC) 112. The transducer 110 may be positioned on a lower surface of the ultrasonic diagnosis apparatus 10, transmit ultrasonic waves to an object in contact therewith, and receive echo signals reflected from the object.

More specifically, the transducer 110 may include the transducer element 111 that generates ultrasonic waves. The transducer element 111 may include a magnetostrictive ultrasonic transducer using a magnetostrictive effect of a magnetic material used in an ultrasonic probe device, a piezoelectric ultrasonic transducer using a piezoelectric effect of a piezoelectric material, and/or a capacitive micromachined ultrasonic transducer (CMUT) that transmits and receives ultrasonic waves using vibrations of several hundreds or thousands of micromachined thin films.

In addition, the transducer 110 may include the application specific integrated circuit (ASIC) 112 in which a CMUT array is bonded in a flip chip bonding method. The signal line of the ASIC 112 in which the CMUT array is bonded may be bonded to a board by a wire bonding method, or the ASIC 112 may be electrically connected to the board through a flexible printed circuit board. The board may include a transmitter, and when electrical signals are applied through the transmitter of the board, the electrical signals applied to the CMUT array may be controlled in accordance with logic of the ASIC 112 to thereby adjust generation of ultrasonic waves.

The beamformer 120 may perform beamforming on ultrasonic echo signals output from the transducer 110. The beamformer 120 may include an analog to digital (AD) converter that converts the ultrasonic echo signals into digital signals, and a digital beamformer 122 that performs beamforming on the ultrasonic echo signals converted into the digital signals output from the AD converter 121.

More specifically, the AD converter 121 may receive successive ultrasonic echo signals from the transducer 110, and convert the received echo signals into digital signals. In this instance, the same number of AD converters 121 as the number of channels may be provided.

In addition, the beamformer 122 may perform beamforming. Here, beamforming refers to an operation of inputting signals of a plurality of channels, for example, a plurality of echo signals, from a target portion, correcting a time difference of the input signals of each channel, and emphasizing or attenuating signals of a specific channel by assigning a predetermined weight to each of the signals whose time differences are corrected, thereby focusing the signals of the plurality of channels.

More specifically, echo ultrasonic waves reflected and returned from the same target portion may have different times during which the transducer element 111 receives the echo ultrasonic waves. That is, in the reception of each of the echo ultrasonic waves of the same target portion, a predetermined time difference may be present. This is because not all distances between the target portion and elements constituting the transducer element 111 that receives the echo ultrasonic waves are the same. Thus, the echo ultrasonic waves received by the respective elements at different times may be the echo ultrasonic waves reflected and returned from the same target portion. Thus, the beamformer 122 corrects the time difference between the ultrasonic signals. For example, the echo ultrasonic wave input through a specific channel is delayed at a constant level to correct the time difference, and the ultrasonic wave whose time difference is corrected is focused.

In addition, the beamformer 122 may focus the echo signals whose time differences are corrected. That is, signals of a specific position are emphasized or attenuated by assigning a predetermined weight to the signals whose time differences are corrected, thereby focusing the signals of the plurality of channels. Thus, it is possible to generate the ultrasonic image in accordance with a user's needs or convenience.

In addition, the signals which are focused and output by the beamformer 122 may be transmitted to the portable terminal 20 through the communicator 130. To this end, the beamformer 122 may assign a different weight for each pixel of the ultrasonic image so that the output signals can be efficiently transmitted to the portable terminal 20.

The ultrasonic echo signals output from the transducer 110 are converted into the digital signals and beamforming is performed by the digital beamformer 122, but exemplary embodiments are not limited thereto. For example, the beamformer 120 may include an analog beamformer, and analog beamforming may be performed by the analog beamformer.

That is, when the analog beamformer receives the ultrasonic echo signals output from the transducer 110 to correct the time difference and the AD converter 121 converts the ultrasonic echo signals whose time difference is corrected into digital signals, the digital beamformer 122 may focus the converted ultrasonic echo signals.

In addition, the beamformer 120 including the analog beamformer and/or the digital beamformer 122 and the AD converter 121 may be implemented in a single chip and provided in the ultrasonic diagnosis apparatus 10.

The communicator 130 may exchange signals of the ultrasonic diagnosis apparatus 10 and the portable terminal 20. That is, the communicator 130 may transmit, to the portable terminal 20, data signals or control signals such as the output signals output from the beamformer 120, or receive the data signals or the control signals from the portable terminal 20.

More specifically, the communicator 130 may include a communicator 131 and a port 132. The communicator 131 may convert the data signals output from the beamformer 120 or the control signals generated by the controller 140 into the type of signals that can be transmitted to the portable terminal 20. That is, the communicator 131 may determine a transmission scheme, and convert the control signals into the type of the signals in accordance with the determined transmission scheme, thereby transmitting and/or receiving various kinds of signals to and/or from the portable terminal 20 through the port 132.

In addition, in the communicator 131, a transmission method of the data signals and a transmission method of the control signals may be different from each other. For example, when the port 132 is a universal serial bus (USB), the signals on which beamforming is performed may be output using a bulk transfer scheme, and the control signals may be transmitted and received using a control transfer scheme.

In addition, the communicator 131 may encode data or control signals to be transmitted to the portable terminal 20, or decode the data or control signals transmitted from the portable terminal 20. For example, the communicator 130 may encode the data signals output from the beamformer 120 to transmit the encoded data signals to the portable terminal 20, or decode the signals received from the portable terminal 20 to transmit the decoded signals to the controller 140.

The ultrasonic diagnosis apparatus 10 and the portable terminal 20 may be docked together through the port 132 of the ultrasonic diagnosis apparatus 10 and the terminal port 211 of the portable terminal 20, respectively. In addition, signals between the ultrasonic diagnosis apparatus 10 and the portable terminal 20 may be exchanged through the port 132 and the terminal port 211.

More specifically, The port 132 may be provided in the form of a socket or a plug to be docked with the terminal port 211. For example, the port 132 protrudes in the form of the plug and the terminal port 211 is provided in the form of the socket so that the ultrasonic diagnosis apparatus 10 and the portable terminal 20 can be docked together, or the port 132 is provided in the form of the socket and the terminal port 211 protrudes in the form of the plug so that the ultrasonic diagnosis apparatus 10 and the portable terminal 20 can be docked together.

In addition, the port 132 may be provided to conform to the terminal port 211. For example, when the terminal port 211 is the USB terminal, the port 132 of the ultrasonic diagnosis apparatus 10 may be provided in the form of a terminal that can be coupled to the USB terminal to allow the ultrasonic diagnosis apparatus 10 and the portable terminal 20 to be docked together, and when the terminal port 211 is a mini-USB terminal, the port 132 of the ultrasonic diagnosis apparatus 10 may be provided in the form of a terminal that can be coupled to the mini-USB terminal to allow the ultrasonic diagnosis apparatus 10 and the portable terminal 20 to be docked together.

In addition, the port 132 may receive power from the portable terminal 20. That is, the ultrasonic diagnosis apparatus 10 may be operated by receiving power from the battery 250 of the portable terminal 20. In this manner, by supplying the power to the ultrasonic diagnosis apparatus 10 using the battery 250 provided in the portable terminal 20, the volume of the ultrasonic diagnosis apparatus 10 may be reduced to increase mobility.

In the above, a case in which the communicator 131 transmits and/or receives signals to and/or from the portable terminal 20 through the port 132 has been described, but the communicator 130 may transmit and/or receive signals to and/or from the portable terminal 20 in a different scheme.

More specifically, the communicator 131 may exchange signals with the portable terminal 20 in accordance with a wireless communication scheme. For example, the communicator 131 may exchange signals with the portable terminal 20 using a mobile communication protocol such as global system for mobile communications (GSM), code division multiple access (CDMA), wideband code division multiple access (WCDMA), time division multiple access (TDMA), long term evolution (LTE), or the like, or a short-range communication protocol such as wireless local access network (WLAN), Bluetooth, Zigbee, or the like.

In addition, the communicator 131 may use a communication method through the port 132 together with a different communication method. For example, the communicator 131 may transmit and/or receive control-related signals to and/or from the portable terminal 20 in accordance with the wireless communication scheme, and transmit and/or receive data signals such as beamforming signals to and/or from the portable terminal 20 through the port 132.

In this manner, by using a combination of the communication scheme through the port 132 and the wireless communication scheme, transmission efficiency of signals may be increased. Thus, it is possible to output ultrasonic images in real-time, and acquire ultrasonic images with higher image quality.

The controller 140 may control the overall operations of the ultrasonic diagnosis apparatus 10.

More specifically, the controller 140 may control the transducer 110 to generate ultrasonic waves and transmit the generated ultrasonic waves to an object. In addition, the controller 140 may adjust the ultrasonic waves generated in the transducer 110 by controlling the power supplied from the battery 250 of the portable terminal 20.

In this manner, by receiving the power from the battery 250 of the portable terminal 20, the ultrasonic diagnosis apparatus 10 may be further miniaturized to reduce production costs of the ultrasonic diagnosis apparatus 10.

In addition, the controller 140 may control beamforming of the beamformer 120. For example, the controller 140 may control the beamformer 120 to generate output signals by performing beamforming on the echo signals with a magnitude that can be transmitted to the portable terminal 20 in real-time. Thus, the controller 140 may enable the communication terminal to generate the ultrasonic images in real-time by controlling performance of beamforming in accordance with performance of each communicator 130.

In addition, the controller 140 may enable the portable terminal 20 and the ultrasonic diagnosis apparatus 10 to transmit and receive various signals by controlling the communicator 130. More specifically, the controller 140 may transmit signals on which beamforming has been performed through the communicator 130. In addition, the controller 140 may transmit control signals related to image processing, ultrasonic image output, and the like through the communicator 130 to control the portable terminal 20.

In this manner, by the ultrasonic diagnosis apparatus 10 performing image processing using resources of the portable terminal 20, the ultrasonic diagnosis apparatus 10 need not include separate hardware and/or software for image processing. Thus, it is possible to miniaturize the ultrasonic diagnosis apparatus 10 and reduce production costs of the ultrasonic diagnosis apparatus 10.

In addition, the ultrasonic diagnosis apparatus 10 may enable the ultrasonic images to be output in real-time in the portable terminal 20 by transmitting and receiving the signals on which beamforming has been performed to and from the portable terminal 20 through the port 132.

In addition, the ultrasonic diagnosis apparatus 10 may output the ultrasonic images using a terminal display 230 of the portable terminal 20 without including a separate display for outputting the ultrasonic images in the ultrasonic diagnosis apparatus 10, and therefore it is possible to miniaturize the ultrasonic diagnosis apparatus 10 and reduce the production cost of the ultrasonic diagnosis apparatus 10.

In addition, the controller 140 may receive control signals of a user from the portable terminal 20. The user may input various commands for controlling the ultrasonic diagnosis apparatus 10 through a terminal input unit 240 of the portable terminal 20. In this manner, when the command is input through the terminal input unit 240 of the portable terminal 20, the portable terminal 20 may generate control signals to transmit the generated control signals through a terminal communicator 210, and the controller 140 may control operations of the ultrasonic diagnosis apparatus 10 in accordance with the control signals received through the communicator 130.

Thus, the ultrasonic diagnosis apparatus 10 may receive the commands from the user through the portable terminal 20 even when the ultrasonic diagnosis apparatus 10 does not include separate hardware for receiving the commands from the user, and therefore it is possible to miniaturize the ultrasonic diagnosis apparatus 10 and reduce the production costs of the ultrasonic diagnosis apparatus 10.

In addition, the controller 140 may recognize the portable terminal 20 when the portable terminal 20 is docked on the port 132. To this end, the controller 140 may further include specific software. More specifically, the controller 140 may perform a series of procedures for recognizing the portable terminal 20 without separate physical settings when the portable terminal 20 is docked on the port 132.

The controller 140 may correspond to one or a plurality of processors. In this instance, the processor may be implemented as an array of a plurality of logic gates, or as a combination of a general-purpose microprocessor and a memory in which a program executable in the general-purpose microprocessor is stored. In addition, the processor may be implemented in different types of hardware, which can be understood by one of ordinary skill in the art.

In addition, in accordance with an exemplary embodiment, a case in which the ultrasonic diagnosis apparatus 10 includes the transducer 110, the beamformer 120, the communicator 131, and the controller 140 has been described, but this is for convenience of description and the exemplary embodiments are not limited thereto. For example, the transducer 110, the beamformer 120, the communicator 131, and the controller 140 may be formed as a single device according to embodiments, or the transducer 110 and the beamformer 120 may be formed as a single device.

The portable terminal 20 may include the terminal communicator 210, an image processor 220, the terminal display 230, the terminal input unit 240, the battery 250, and a terminal controller 260. The portable terminal 20 may output ultrasonic images in accordance with control of the ultrasonic diagnosis apparatus 10.

The portable terminal 20 in accordance with an exemplary embodiment may be any device that can be connected to the ultrasonic diagnosis apparatus 10. For example, the portable terminal 20 may be a mobile terminal such as a laptop, a mobile phone, a portable media player (PMP), a personal digital assistant (PDA), a tablet personal computer (PC), or the like. In an exemplary embodiment, the portable terminal 20 may be a smart phone.

The terminal communicator 210 may enable the portable terminal 20 and the ultrasonic diagnosis apparatus 10 to exchange signals therebetween. That is, the terminal communicator 210 may transmit or receive signals to or from the ultrasonic diagnosis apparatus 10.

More specifically, the terminal communicator 210 may include a terminal communicator 212 and a terminal port 211.

The terminal communicator 212 may determine a transmission scheme for communicating with the ultrasonic diagnosis apparatus 10, convert data signals or control signals into signals that can be transmitted and/or received to and/or from the ultrasonic diagnosis apparatus 10 in accordance with the determined transmission scheme, and transmit and/or receive the data signals or the control signals to and/or from the ultrasonic diagnosis apparatus 10 through the terminal port 211.

In addition, the terminal communicator 212 may convert the output signals or the control signals received from the ultrasonic diagnosis apparatus 10 into signals that can be used in the portable terminal 20, by decoding the output signals or the control signals, and encode various data signals or control signals to transmit the encoded signals to the ultrasonic diagnosis apparatus 10.

The ultrasonic diagnosis apparatus 10 and the portable terminal 20 may be docked together through the terminal port 211. That is, signals may be transmitted and/or received through the port 132 of the docked ultrasonic diagnosis apparatus 10 and the terminal port 211 of the portable terminal 20. More specifically, the terminal port 211 may be provided in the form of a socket or a plug to be docked with the port 132 provided in the form of the plug or the socket.

In addition, the terminal port 211 may supply power to the ultrasonic diagnosis apparatus 10. The portable terminal 20 may provide the power for driving the ultrasonic diagnosis apparatus 10 by transmitting electric energy stored in the battery 250 through the port 132.

While the case in which the terminal communicator 212 transmits and receives signals to and from the ultrasonic diagnosis apparatus 10 through the terminal port 211 has been described, the terminal communicator 212 may transmit and receive signals to and from the portable terminal 20 in a different scheme.

More specifically, the terminal communicator 212 may exchange signals with the ultrasonic diagnosis apparatus 10 in accordance with a wireless communication scheme. For example, the terminal communicator 212 may exchange signals with the ultrasonic diagnosis apparatus 10 using a mobile communication protocol such as GSM, CDMA, WCDMA, TDMA, LTE, or the like, or a short-range communication protocol such as WLAN, Bluetooth, Zigbee, or the like.

In addition, the terminal communicator 212 may use a communication scheme through the terminal port 211 together with a different communication scheme. For example, the terminal communicator 212 may transmit and receive control-related signals to and from the ultrasonic diagnosis apparatus 10 in accordance with the wireless communication scheme, and transmit and receive data signals such as beamforming signals to and from the ultrasonic diagnosis apparatus 10 through the terminal port 211.

In this manner, by using a combination of the communication scheme through the port 132 and the wireless communication scheme, transmission efficiency of data signals may be increased. Thus, it is possible to output ultrasonic images in real-time, and acquire ultrasonic images with higher image quality.

The image processor 220 may generate ultrasonic images based on output signals received from the terminal communicator 210. In this instance, the ultrasonic images may be generated in various modes. For example, the ultrasonic images may be generated in an A-mode in which intensity of echo signals is represented as a size of amplitude, a B-mode in which the ultrasonic images are converted into brightness or luminance to be represented, an M-mode in which a distance with a moving inspection portion of an object is represented as a temporal change, a D-mode in which pulse waves or continuous waves are used, a color flow mapping (CFM)-mode in which the ultrasonic images are represented as color images using the Doppler effect, or the like.

In addition, the image processor 220 may further perform separate additional image processing on the restored ultrasonic images. For example, the image processor 220 may further perform image post-processing such as correcting or re-correcting contrast, brightness, or sharpness of the ultrasonic images.

In this instance, the image processor 220 may perform image processing so that a part of the generated ultrasonic images can be emphasized or attenuated. In addition, when a plurality of ultrasonic images are generated, the image processor 220 may generate three-dimensional (3D) ultrasonic images using the plurality of ultrasonic images.

In this manner, the additional image processing of the image processor 220 may be performed in accordance with a predetermined setting, and may be further performed in accordance with commands of the user input through the terminal input unit 240.

The terminal display 230 may display a variety of information related to the portable terminal 20, or output the ultrasonic images or information related to setting of the ultrasonic image apparatus 10.

In this instance, the terminal display 230 may be implemented in, for example, a liquid crystal display (LCD), a light emitting diode (LED), an organic light emitting diode (OLED), an active matrix organic light emitting diode (AMOLED), a flexible display, a 3D display, or the like, and when the terminal display 230 includes a touch screen, the terminal display 230 may also perform a function corresponding to the terminal input unit 240.

The terminal input unit 240 may transmit various electric signals input by the user to the controller 140, and may be implemented as various types of input means. For example, the terminal input unit 240 may be a gesture input unit or a voice input unit as well as different types of an input unit such as a key input unit (e.g., a keyboard), a touch sensor, a touch pad, or the like.

The battery 250 may store electric energy for driving the ultrasonic image apparatus or the portable terminal 20. That is, in the battery 250, chemical energy that can be converted into electric energy may be stored.

In this instance, the battery 250 may be a primary battery or a secondary battery that is reusable through charging. For example, the battery 250 may be a lithium battery or a lithium polymer battery that is reusable through charging.

The terminal controller 260 may control the overall operations of the portable terminal 20.

More specifically, the terminal controller 260 may control the image processor 220 to perform image processing in accordance with control signals of the ultrasonic diagnosis apparatus 10 so that the ultrasonic images can be output to the terminal display 230.

In addition, the terminal controller 260 may control the terminal display 230 to output the ultrasonic images based on the data processed by the image processor 220.

In addition, the terminal controller 260 may provide the electric energy stored in the battery 250 to the ultrasonic diagnosis apparatus 10 through the terminal port 211 in accordance with the control signals of the ultrasonic diagnosis apparatus 10.

In addition, the terminal controller 260 may control the portable terminal 20 to be operated in accordance with the control signals input by the user through the terminal input unit 240. In addition, the terminal controller 260 may transmit the control signals input by the user through the terminal input unit 240 to the ultrasonic diagnosis apparatus 10.

The terminal controller 260 may correspond to one or a plurality of processors. In this instance, the one or the plurality of processors may be built in the portable terminal 20. In addition, the image processor 220 and the controller 140 have been described as being separate from each other, but may be implemented by a single processor.

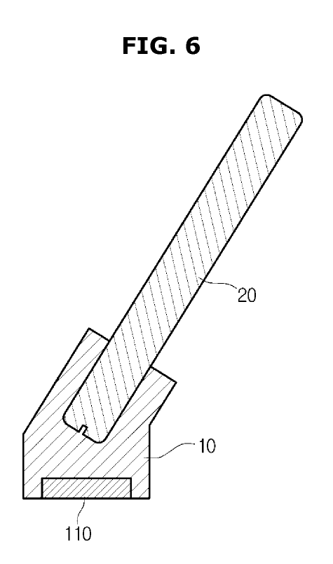

FIG. 6 is a cross-sectional view showing a medical system according to another exemplary embodiment.

Referring to FIG. 6, the ultrasonic diagnosis apparatus 10 may have various shapes for the user's convenience. More specifically, in order for the ultrasonic images output by the portable terminal 20 to be readily observed by a user, the ultrasonic diagnosis apparatus 10 may have a shape such that the docked portable terminal 20 can form a predetermined angle with the transducer 110.

For example, as shown in FIG. 6, the docked portable terminal 20 has a structure in which the docked portable terminal 20 forms the predetermined angle with the transducer 110 of the ultrasonic diagnosis apparatus 10, whereby a user can readily observe the ultrasonic images displayed on the docked portable terminal 20.

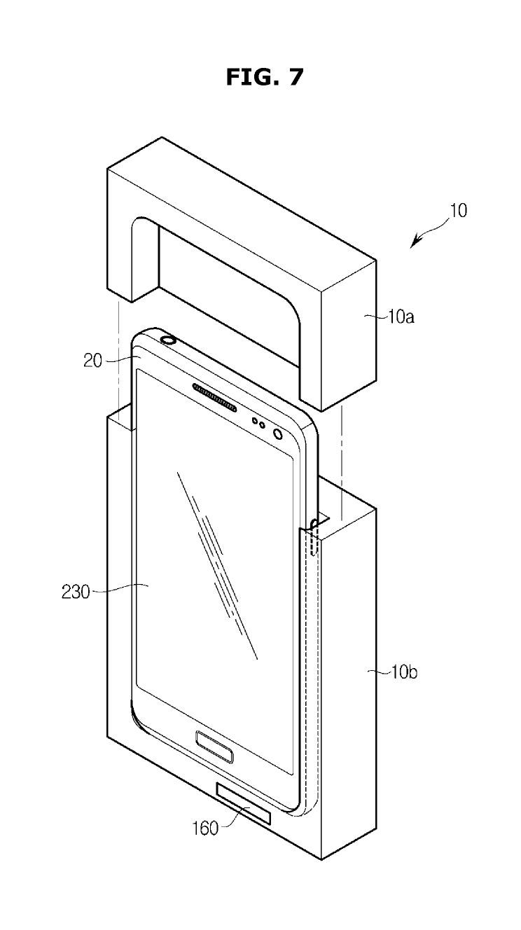

Hereinafter, a medical system in accordance with another exemplary embodiment will be described in detail with reference to FIGS. 7 to 10. Hereinafter, the same reference numerals are used to refer to the same elements, and thus the repetitive descriptions thereof will be omitted.

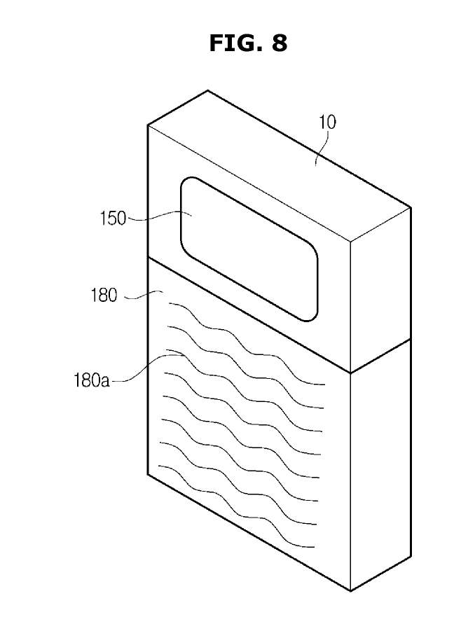

FIG. 7 is a perspective view showing a front side of a medical system in accordance with another exemplary embodiment, and FIG. 8 is a perspective view showing a rear surface of a medical system in accordance with another exemplary embodiment.

Referring to FIGS. 7 and 8, the portable terminal 20 may be docked on the ultrasonic diagnosis apparatus 10. More specifically, the ultrasonic diagnosis apparatus 10 may have a space therein in which the portable terminal 20 can be seated. In this manner, the portable terminal 20 may be seated on the space in the ultrasonic diagnosis apparatus 10 so that the portable terminal 20 and the ultrasonic diagnosis apparatus 10 may be docked together.

In this instance, the ultrasonic diagnosis apparatus 10 may be divided into an upper portion 10a and a lower portion 10b.

For example, as shown in FIG. 7, the ultrasonic diagnosis apparatus 10 may be divided into the upper portion (i.e., a first housing) 10a and the lower portion (i.e., a second housing) 10b, and the portable terminal 20 may be docked on the ultrasonic diagnosis apparatus 10, which is separated into the upper portion 10a and the lower portion 10b.

In addition, by separating the ultrasonic diagnosis apparatus 10 into the upper portion 10a and the lower portion 10b, the portable terminal 20 seated on the ultrasonic diagnosis apparatus 10 may be separated from the ultrasonic diagnosis apparatus 10.

The terminal port 211 may be provided on an upper portion 10a of the ultrasonic diagnosis apparatus 10. In this instance, the lower portion 10b of the ultrasonic diagnosis apparatus 10 may receive the transducer 110, the beamformer 120, and the like.

Thus, even when the port 132 of the portable terminal 20 is changed, only the upper portion 10a of the ultrasonic diagnosis apparatus 10 may be replaced. That is, the portable terminal 20 may have various ports 132 (for example, a mini-USB a type or a mini-USB b type). Thus, to increase utilization of the ultrasonic diagnosis apparatus 10, docking with the portable terminal 20 having the various ports 132 may be supported.

Therefore, devices which are not affected by the port 132 of the portable terminal 20 such as the transducer 110, the beamformer 120, and the like may be received in the lower portion 10b of the ultrasonic diagnosis apparatus 10, and the communicator 130 that is affected by the port 132 of the portable terminal 20 may be received in the upper portion 10a of the ultrasonic diagnosis apparatus 10. Accordingly, even when the port 132 of the portable terminal 20 is changed, only the upper portion 10a of the ultrasonic diagnosis apparatus 10 may be replaced, thereby increasing the utilization of the ultrasonic diagnosis apparatus 10.

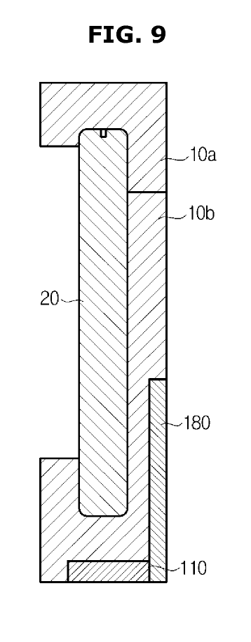

FIG. 9 is a cross-sectional view showing a medical system in accordance with another exemplary embodiment.

Referring to FIGS. 7 to 9, the ultrasonic diagnosis apparatus 10 may further include a display 150. In this instance, the display 150 may output ultrasonic images or screens for setting the ultrasonic diagnosis apparatus 10 or adjusting the setting of the ultrasonic diagnosis apparatus 10.

For example, the display 150 may be implemented as a display means such as an LCD, an LED, an OLED, an AMOLED, a flexible display, a 3D display, or the like.

More specifically, the display 150 may output the ultrasonic images in accordance with data on which image processing has been performed by the portable terminal 20, or display a variety of information related to the ultrasonic diagnosis apparatus 10. In this manner, by performing image processing using the resources of the portable terminal 20, the ultrasonic diagnosis apparatus 10 may be miniaturized and production costs of the ultrasonic diagnosis apparatus 10 may be reduced.

The display 150 may be used as an auxiliary output device. For example, when the portable terminal 20 is docked on the ultrasonic diagnosis apparatus 10 so that the ultrasonic images are generated through the portable terminal 20, the display 150 may output no ultrasonic image or may display only information related to setting of the ultrasonic diagnosis apparatus 10.

The ultrasonic diagnosis apparatus 10 may further include an input unit 160 for receiving commands from the user. In this instance, the input unit 160 may receive a predetermined command from the user, and generate control signals corresponding to the received command to transmit the generated control signals to the controller 140.

Commands may be input through the input unit 160, and may be also input through the terminal input unit 240 of the docked portable terminal 20. For example, the input unit 160 may receive only on/off commands of the ultrasonic diagnosis apparatus 10 or a recognition command of the portable terminal 20, and other commands, e.g., commands related to the generation of the ultrasonic images may be received through the terminal input unit 240.

In addition, when the display 150 includes a touch screen, the display 150 may also perform a function of the input unit 160.

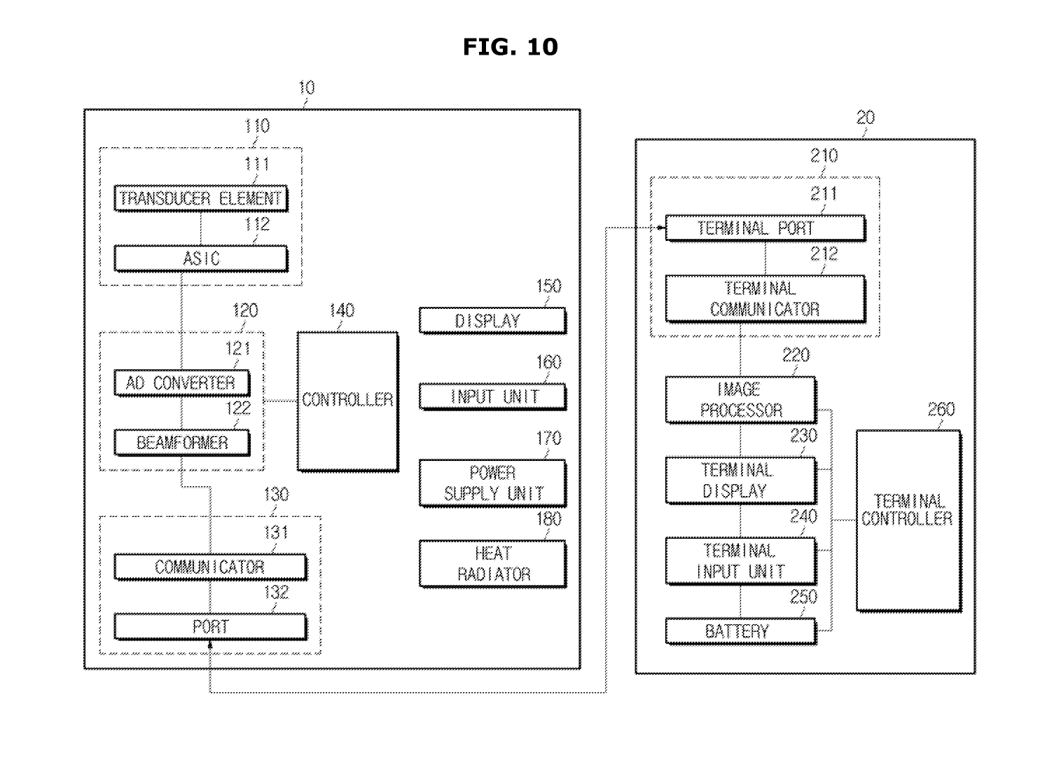

The ultrasonic diagnosis apparatus 10 may further include a power supply unit 170 (see FIG. 10). The power supply unit 170 may supply power for driving the ultrasonic diagnosis apparatus 10. In this instance, the power supply unit 170 may supply the power for driving the ultrasonic diagnosis apparatus 10 by receiving power from the outside, or based on the electric energy stored inside the ultrasonic diagnosis apparatus 10 such as a battery.

The power supply unit 170 of the ultrasonic diagnosis apparatus 10 may use the battery 250 of the portable terminal 20 as auxiliary power. For example, the power for driving the ultrasonic diagnosis apparatus 10 may be supplied through the power supply unit 170 of the ultrasonic diagnosis apparatus 10, and when it is difficult to supply the power from the power supply unit 170, the power for driving the ultrasonic diagnosis apparatus 10 may be supplied through the battery 250 of the portable terminal 20.

In this manner, the power for driving the ultrasonic diagnosis apparatus 10 may be further supplied by the battery 250 of the portable terminal 20, and therefore it is possible to increase the life of the ultrasonic diagnosis apparatus 10.

The ultrasonic diagnosis apparatus 10 may further include a heat radiator 180. As the ultrasonic diagnosis apparatus 10 is miniaturized, performance of the ultrasonic diagnosis apparatus 10 may be deteriorated by the heat generated in the ultrasonic diagnosis apparatus 10, or durability of the ultrasonic diagnosis apparatus 10 may be reduced due to the heat generated in the ultrasonic diagnosis apparatus 10.

For example, when the number of the transducer elements 111 provided in the ultrasonic diagnosis apparatus 10 is increased, heat generation of the ultrasonic diagnosis apparatus 10 may increase. In addition, as the degree of intensity or the performance of the beamformer 120 or the controller 140 is increased, the heat generation may increase accordingly. In this manner, when the heat generation becomes excessive due to the driving of the ultrasonic diagnosis apparatus 10, performance of the ultrasonic diagnosis apparatus 10 may be deteriorated.

Thus, the ultrasonic diagnosis apparatus 10 may further include the heat radiator 180 to emit heat generated by the driving of the ultrasonic diagnosis apparatus 10 to the outside. In this instance, the heat radiator 180 may emit the heat generated in the ultrasonic diagnosis apparatus 10 to the outside using, for example, a fluid such as water or air in the atmosphere.

As shown in FIG. 8, the heat radiator 180 may be provided on a surface of the ultrasonic diagnosis apparatus 10. In this instance, the heat radiator 180 may emit the heat generated by the driving of the ultrasonic diagnosis apparatus 10 to the outside by conducting the heat.

More specifically, a surface of the heat radiator 180 may be in tight contact with devices that emit heat such as the beamformer 120, the transducer 110, and the controller 140, and another surface of the heat radiator 180 may be brought into contact with external air. In this manner, by conducting the external heat of the ultrasonic diagnosis apparatus 10 to the outside, the heat generated by the driving of the ultrasonic diagnosis apparatus 10 may be emitted to the outside.

In addition, the heat radiator 180 may have a corrugated groove 180a to increase a contact area with the air. In this manner, the contact area with the air is increased by the corrugated groove 180a, thereby further increasing the emission effect of the heat.

In addition, the heat radiator 180 may include a material that has higher thermal conductivity and is durable against heat. For example, the heat radiator 180 may include a material such as aluminum, pure copper, brass, bronze, ceramics, or the like.

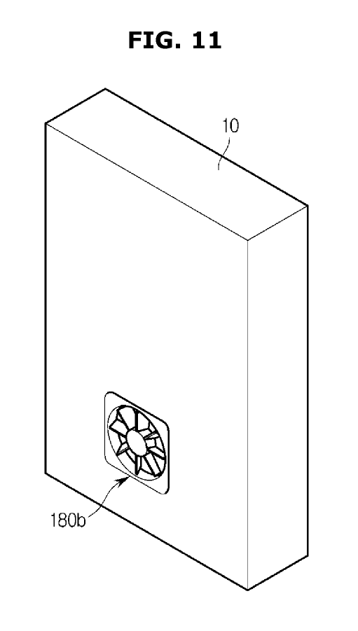

FIG. 10 is a control block diagram showing a medical system in accordance with another exemplary embodiment. FIG. 11 is a perspective view showing another example of a heat radiator.

Referring to FIG. 11, the heat radiator 180 may further include an air fan 180b. In this instance, the air fan 180b may emit the air inside the ultrasonic diagnosis apparatus 10 to the outside to thereby emit the heat generated in the ultrasonic diagnosis apparatus 10 to the outside. In this instance, the air fan 180b may receive driving power from the power supply unit 170 or the battery 250.

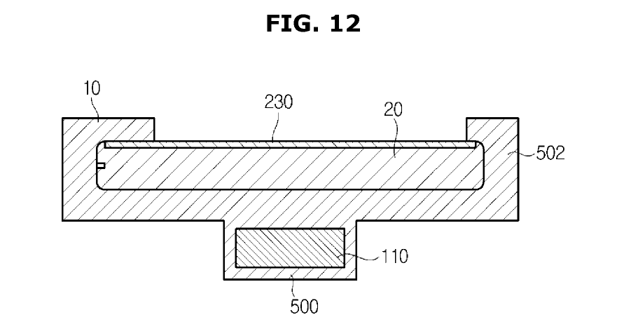

FIG. 12 is a schematic cross-sectional view showing a medical system cut along a horizontal direction in accordance with an exemplary embodiment.

Referring to FIG. 12, the transducer 110 may be located on a first side 500 of the ultrasonic diagnosis apparatus 10, which is opposite to a second side 502 of the ultrasonic diagnosis apparatus 10 on which the portable terminal 20 is docked with the ultrasonic diagnosis apparatus 10. The ultrasonic images may be output through the terminal display 230. In this instance, the transducer 110 may be provided on the opposite side of the terminal display 230 of the portable terminal 20.

In this manner, when the transducer 110 is provided on the opposite side of the terminal display 230, the ultrasonic images may be more readily observed. That is, it is possible to prevent the ultrasonic images from being covered by the hand of the user.

As described above, in accordance with exemplary embodiments, image processing may be performed on the signals subject to beamforming using hardware of the portable terminal, and the ultrasonic signals subject to image processing may be output, thereby miniaturizing the ultrasonic diagnosis apparatus.

In addition, according to exemplary embodiments, the ultrasonic images may be output using the processor and the display of the portable terminal, thereby reducing the production costs of the ultrasonic diagnosis apparatus.

In addition, according to exemplary embodiments, the ultrasonic images may be generated using the battery provided in the portable terminal, thereby increasing the life of the ultrasonic diagnosis apparatus, and reducing the production costs of the ultrasonic diagnosis apparatus.

In addition, according to exemplary embodiments, the ultrasonic diagnosis apparatus including the heat radiator that can emit the heat generated by the driving of the ultrasonic diagnosis apparatus may be provided, thereby improving thermal stability of the ultrasonic diagnosis apparatus.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An ultrasonic diagnosis apparatus comprising:
a transducer configured to transmit ultrasonic waves to an object and receive echo signals reflected from the object;
a beamformer configured to generate output signals by performing beamforming on the echo signals;
a port configured to engage with a portable terminal; and
a controller configured to control an ultrasonic image to be displayed on the portable terminal by transmitting the output signals to the portable terminal engaged with the port.

2. The ultrasonic diagnosis apparatus according to claim 1, wherein the controller is configured to control the portable terminal to receive the output signals, and control the portable terminal to perform image processing on the received output signals.

3. The ultrasonic diagnosis apparatus according to claim 1, wherein the controller is configured to control the portable terminal so that the ultrasonic image is displayed on a display provided in the portable terminal.

4. The ultrasonic diagnosis apparatus according to claim 1, wherein the controller is configured to transmit the output signals to the portable terminal through the port.

5. The ultrasonic diagnosis apparatus according to claim 1, wherein the controller is configured to transmit the output signals to the portable terminal wirelessly.

6. The ultrasonic diagnosis apparatus according to claim 1, further comprising:
a first housing in which the port is provided; and
a second housing configured to receive the transducer, the beamformer, and the controller,
wherein a space in which the port is engaged with the portable terminal is formed by coupling the first housing and the second housing.

7. The ultrasonic diagnosis apparatus according to claim 5, wherein the first housing is replaceable depending on a terminal port of the portable terminal.

8. The ultrasonic diagnosis apparatus according to claim 1, further comprising:
a fixer configured to fix the portable terminal engaged with the port.

9. The ultrasonic diagnosis apparatus according to claim 1, further comprising:
a heat radiator configured to emit heat generated by driving the ultrasonic diagnosis apparatus to an outside.

10. The ultrasonic diagnosis apparatus according to claim 9, wherein the heat radiator configured to emit the generated heat to the outside via a heat conduction.

11. The ultrasonic diagnosis apparatus according to claim 9, wherein the heat radiator includes a cooling fan, and is configured to emit the generated heat to the outside by rotating the cooling fan.

12. The ultrasonic diagnosis apparatus according to claim 1, wherein the portable terminal engaged with the port is configured to supply power for driving the ultrasonic diagnosis apparatus through the port.

13. The ultrasonic diagnosis apparatus according to claim 1, further comprising:
a power supply unit configured to supply power for driving the ultrasonic diagnosis apparatus.

14. The ultrasonic diagnosis apparatus according to claim 1, wherein the portable terminal is configured to engage with the ultrasonic diagnosis apparatus to have an angle with respect to the transducer.

15. The ultrasonic diagnosis apparatus according to claim 1, wherein the transducer is located on a first side of the ultrasonic diagnosis apparatus opposite to a second side on which the portable terminal is disposed.

16. The ultrasonic diagnosis apparatus according to claim 1, wherein the controller is configured to control operations of the ultrasonic diagnosis apparatus in accordance with control signals received from the portable terminal engaged with the port.
